(19) United States
(12) Patent Application Publication
(10) Pub. No.: US 2009/0062680 A1
(43) Pub. Date: Mar. 5, 2009

(54) ARTIFACT DETECTION AND CORRECTION SYSTEM FOR ELECTROENCEPHALOGRAPH NEUROFEEDBACK TRAINING METHODOLOGY

(75) Inventor: JOSEPH A. SANDFORD, Richmond, VA (US)

Correspondence Address: Thomas & Raring, P.C., 536 GRANITE AVENUE, RICHMOND, VA, 23226, US

(73) Assignee: Brain Train, Richmond, VA (US)

(21) Appl. No.: 12/198,972

(22) Filed: Aug. 27, 2008

Related U.S. Application Data
(60) Provisional application No. 60/969,891, filed on Sep. 4, 2007.

(51) Int. Cl. A61B 5/0476

(52) U.S. Cl. 600/544

(57) ABSTRACT

The method for simultaneously and concurrently identifying and quantifying a wide variety of types of facial electromyographic (EMG) and eye movement electrooculargraphic (EOG) activity, which naturally contaminate electroencephalographic (EEG) waveforms in order to significantly improve the accuracy of the calculation in real-time of the amplitude and/or coherence of any brainwave activity for any chosen frequency bandwidth for any number of electrode placements. This multi-level, widely or universally applicable, pre-defined pattern recognition artifact detection and correction system provides a method for enhancing EEG biofeedback training by detecting and eliminating any brief, contaminated epoch of EEG activity from being included in the calculation and analysis of the EEG signal. The method and apparatus disclosed herein make it possible to provide without any interruption visual, auditory and/or tactile feedback of a “true” EEG signal that through operant conditioning learning principles enables individuals to more quickly and easily learn to control their brainwave activity using neurofeedback.

This application claims the benefit of provisional application No. 60/969,891 filed on Sep. 4, 2007, which is incorporated by reference herein

### FIELD OF THE INVENTION

This invention involves a method for the identification and correction of artifacts that impair the accurate calculation, analysis and presentation of feedback of electroencephalography (EEG) signals used in the provision of neurofeedback training. This method involves the simultaneous and concurrent identification and detection in real-time of a variety of different types of electro-oculographic (EOG), electromyographic (EMG) and related muscular and/or environmentally generated artifacts that spontaneously and/or volitionally occur that impair neurofeedback training particularly in the frontal and temporal lobe regions of the brain where EOG and EMG artifact activity is often more prevalent.

### BACKGROUND

In general, neurofeedback is best understood as a training process which involves measuring a person's brainwave activity and then communicating specific information to him or her in real-time so that an individual can become more aware of the psychophysiological processes of one or more selected brain areas. The purpose of neurofeedback training is to enable individuals to learn how to gain conscious control of specific brainwave frequency patterns and/or change the interaction and communication between the different functional centers of their brain. This increase in a personas control of their brainwave functioning has been found in a large number of scientific studies to lead to improvements for many individuals in respect to their self-regulation or in the reduction of symptoms that negatively impact their quality of life. An annotated bibliography of most of these research studies is available online at www.isnr.org.

As an example, some young people diagnosed with Attention-Deficit/Hyperactivity Disorders (ADD/ADHD) who were described by their parents and teachers as being hyperactive have been found in a number of scientific studies to significantly reduce their hyperactive behavior after neurofeedback training. In many clinical treatment cases, neurofeedback training has typically involved increasing the brainwave frequency defined as the Sensorimotor Rhythm (SMR) in the central area of the brain called the primary motor cortex. Based on behavioral and operant learning theory, the two essential factors that are required in order for neurofeedback training to succeed is that individual being trained needs both accurate information about when the targeted brainwave activity is and is not manifesting and a measure of its signal strength (e.g., its amplitude in microvolts), coherence (i.e., relationship to other signals in different brain sites), or other different types of mathematical measures of signal activity (e.g., increased signal amplitude for a specified EEG frequency bandwidth in one brain region and a simultaneous decreased signal amplitude for a specified EEG frequency bandwidth in different brain region).

It is thought that neurofeedback training in the frontal lobe region is important based on SPECT scan work performed by Dr. Amens. Dr. Amens identified five specific subtypes of ADD/ADHD. Three of these subtypes involved either under- or over-activation in the anterior front lobe regions. Joel and Judith Lubar concluded, after over 20 years of clinical research, that:
“If the greatest amount of dysfunction is in the orbito frontal cortex, the logical locations for recording this activity would be Fp1 and Fp2 [i.e., left and right middle forehead directly above the eyes]; however, eye roll, blink, and frontal EMG artifacts make these sites virtually impossible to use.”
   Thus, the Lubars concluded that this research and many other studies clearly supported that the best location for neurofeedback training would be in the frontal lobe region, but that due to the prevalence of EOG and EMG artifacts, it was virtually impossible to implement any neurofeedback training in the frontal lobe regions.

Since the electrical activity of the brain consists of very small amplitude signals measured in microvolts (i.e., one millionth of a volt), the accurate measurement of brain activity is by nature highly vulnerable to significant distortion and/or complete corruption from both the various natural electrical activities in a person's body (e.g., facial muscles) and from nearby environmental electrical activity such as the powerful electrical motors used in elevators. In general, neurofeedback EEG recording devices use special filters to detect and filter out most environmental electrical “noise” such that this type of artifact does not corrupt or distort the collection of accurate EEG data. However, significant problems remain in respect to muscular and ocular generated artifacts, which significantly distort and impair the accurate measurement and calculation of the EEG signal from various brain locations. These artifacts inherently occur due to the biological fact that electromyographic (EMG) and electroocular (EOG) activities generate signal amplitudes that are in general about 1,000 times or more strong than EEG activity. In other words, it is not easy to accurately measure selected EEG activity when a person blinks, looks around the room, swallows, yawns, grins, grimaces or frowns.

In the above examples, the problem is that there is just too much electrical noise which prevents an accurate measurement of almost all types of EEG activity, particularly in the frontal areas of the brain (i.e., around the forehead region and directly above the eyes). It is for this reason that baseline measures of “normal” EEG activity have mainly been collected only under eyes-closed conditions and for very brief time periods. By this technique, normative EEG signals are relatively artifact free. However, it is recognized that most neurofeedback training needs to be conducted under a more active and alert state with the person's eyes open in order for training to effectively generalize to a person's activities in daily life.

With training occurring with both eyes open, and to a lesser degree with both eyes closed, the accurate measurement of the EEG is well known to be prone to contamination of the signal by facial muscle movements such as the raising of the eyebrows and the furrowing of the brow. Involuntary movements such as blinking and the movement of the eyes can also cause surges in amplitude due to the relatively high amplitude strength of the EMG electrical signal in comparison to the EEG. This artifact problem can lead to false reward presentations for the trainee, which impairs learning. In more extreme cases, an individual can be accidentally trained to increase facial tension in order to achieve what is mistakenly classified as a desired or successful improvement in brain functioning. The identification and correction of artifacts prior to the presentation of a feedback signal helps to insure that the desired brainwave is being trained and not muscle tension or other undesirable artifacts, such as excessively frequent eye blinks or twitches.

Research and development in this area has identified the inherent limitations of prior art in first identifying artifacts and then correcting the EEG signal for optimal use in neurofeedback training. First, the most common method for identifying artifacts is simply to analyze previously recorded EEG data for patterns of artifacts using computer algorithms. This method is based on the traditional approaches used by neurologists who must be trained in this methodology in order, for example, to identify whether or not a patient shows seizure activity in their EEG graph or not. All methods that identify artifacts in this way cannot be used to provide the real-time feedback that is needed for successful neurofeedback training, as discussed above. The second most common method to identify artifacts is to use additional sensors, such as EOG sensors, and measure ocular activity whenever it occurs. In this case, when artifacts do occur, the EEG recording and feedback is either stopped and/or the artifact is flagged by giving the trainee a visual or auditory signal to indicate its occurrence. The limitation of the prior art that uses this method is that all useful EEG signal feedback training is interrupted and stops until the artifact is no longer detected.

In yet another technique, it is known to collect separate, additional artifact signal data using additional sensors, amplifiers, and measuring devices specifically designed for a specific type of artifact In one case EOG artifacts are identified using a plurality of EEG sensory sites for a plurality of sequential epochs using a discriminant function analysis of the cross-correlational, covariational signal data collected based upon a previously collected database of EEG signal data from multiple individuals. In either of these two approaches, an alarm is generated and an attempt is made to correct the EEG signal in real-time (defined as less than 500 ms) by subtracting out the identified artifact signal value. The prior art methodology that uses either of these two methods is inherently limited for two reasons: 1) the EEG signal amplitude cannot be accurately computed, but only approximated, as the additional sensor cannot be located at the same site as the EEG sensor that inherently makes the signal data different, and 2) EMG, and in some cases EOG, data has been found to very closely mimic and mix with EEG signal data making it near impossible to use the “subtract out” method with the any degree of accuracy needed for continuous and artifact free EEG biofeedback training.

The subtraction method can work to some degree with EOG data when there are two sensors and one measures EEG activity and the second EOG artifact activity, except that research by Iwasaki (2005) shows that EOG activity also generates some EMG activity that contaminates the EEG signal. The inherent flaw in this prior art methodology in respect to the accuracy of the realtime calculation of the EEG signal is that any EEG signal measure when artifacts occur is inherently a waveform which contains both the true EEG amplitude signal, as measured in microvolts, and the artifacts of EOG and EMG, which are always in the millivolt range. The subtract out approach will easily result in problems in accuracy as the two types of signals are always mixed when artifacts occur and the artifact amplitude signal is 1,000 or more times stronger than the EEG signal. Bottom line result is that there is a need for a method or system to accurately calculate the EEG signal in real-time using these types of methodologies.

### SUMMARY OF THE INVENTION

The subject method involves the simultaneous and concurrent identification and detection in real-time of a variety of different types of electro-oculographic (EOG), electromyographic (EMG) and related muscular and/or environmentally generated artifacts that spontaneously and/or volitionally occur that impair neurofeed back training particularly in the frontal and temporal lobe regions of the brain where EOG and EMG artifact activity is often more prevalent. When one or more artifacts are detected the measurement and analysis of the EEG is corrected using the methodology described in detail below. The correction provided by the subject methodology occurs in real-time and is completed before the EEG feedback signal is recorded to measure progress or presented to the trainee in visual and/or auditory tactile format. This artifact detection and the subsequent mathematical algorithmic correction of the EEG is designed to provide the participant with more accurate feedback in order to facilitate the speed and ease of this process of the operant learning of EEG brainwave control called neurofeedback.

The subject method involves real-time substitution of last known “good” data readings for data containing detected artifacts. The substitution of good data can occur even during a relatively long period of artifacts, such as when a patient/user smiles. The technique is thought to be more accurate, quicker, and a substantial improvement to a subtraction type methodology.

Some unique and new contributions of this invention include: 1) artifacts are detected in realtime requiring only one EEG sensor using a pre-defined pattern recognition method based on a universally applicable, empirically-derived method that can be adjusted for individual differences and does not require the need for any type of database or correlation computation with any other brain site or the use of any other type of psychophysiological sensor, amplifier, or signal detection device other than an EEG device to be used; 2) in the case of two or more EEG sensor locations, separate artifact detection and corrections are made specifically for each site; 3) the occurrence of a variety of individual and/or combined EOG, EMG, and/or any unusual related or environmental artifacts can be simultaneously and concurrently accurately identified for one or more EEG sensor locations in real-time; and 4) the occurrence of artifact detections and visual, auditory and/or tactile EEG biofeedback is continuously and smoothly displayed without interruption and accurately recorded in real-time by including only epochs of EEG activity that are artifact free in the feedback display and data analysis and recording without the requirement of using any inherently flawed subtraction out methodology.

Basically, the method detects a variety of EMG and EOG artifacts. When the identified artifacts occur the subject method corrects the EEG signal before the person being trained is provided a specific visual or auditory feedback measure of it. Thus, this invention provides a more accurate measure of the targeted EEG activity for data recording and is the basis for generating useful and pleasant feedback signals that facilitate the neurofeedback learning experience. The method incorporates a number of new, unique features not present in prior art, which have the potential to greatly enhance the progress of the field of neurofeedback. The subject method of training is inherently a more pleasant training experience, since the trainee does not have to be concerned or disturbed by their failure to suppress their normally occurring EMG and EOG activity.

It is common for trainees using traditional neurofeedback training to become upset and frustrated when EMG or EOG artifact occurs and they can see or experience through the aberrant feedback and data displayed that these artifacts are distorting and/or corrupting the EEG signal in a variety of ways. The subject method overcomes this shortcoming. Also, the subject method makes it possible for feedback to be more continuous then previously possible, The method also helps to avoid the potential problem of inadvertently training tension in the face that can occur when the neurofeedback training goal is to enhance faster brainwave activity (e.g., beta or 16-21 Hz) in the Frontal Lobe. Previously, tensing of the forehead can produce EMG artifact activity that can be wrongly interpreted as the desired EEG activity, because the conscious or subconscious generation of small amounts of EMG in the face can be falsely interpreted as increases in fast EEG brainwaves. This shortcoming is overcome by the subject method.

Thus, the subject method for artifact detection and correction provides the necessary behavioral learning requirements for neurofeedback as discussed above. As a result, it has the potential to maximize training effectiveness and reduce the training time required to achieve beneficial results.

### BRIEF DESCRIPTION OF THE FIGURES

The foregoing, and additional objects, features, and advantages of the present invention will become apparent to those of skill in the art from the following detailed description of a preferred embodiment thereof, taken in conjunction with the accompanying drawings, in which,

FIG. 1 illustrates a flowchart of one embodiment of the artifact detection and correction system for electroencephalograph neurofeedback training disclosed herein.

### DETAILED DESCRIPTION

The subject method provides for the simultaneous and concurrent 1) identification and 2) detection in real-ime of a variety of different types of electrooculographic (EOG), electromyographic (EMG) and related muscular and/or environmentally generated artifacts that spontaneously and/or volitionally occur that impair neurofeedback training particularly in the frontal and temporal lobe regions of the brain where EOG and EMG artifact activity is often more prevalent. When one or more artifacts are detected, the measurement and analysis of the EEG is corrected. This occurs in real-time and is completed before the EEG feedback signal is recorded to measure progress or presented to the trainee in visual and/or auditory tactile format. This artifact detection and the subsequent mathematical algorithmic correction of the EEG is designed to provide the participant with more accurate feedback in order to facilitate the speed and ease of this process of the operant learning of EEG brainwave control called neurofeedback.

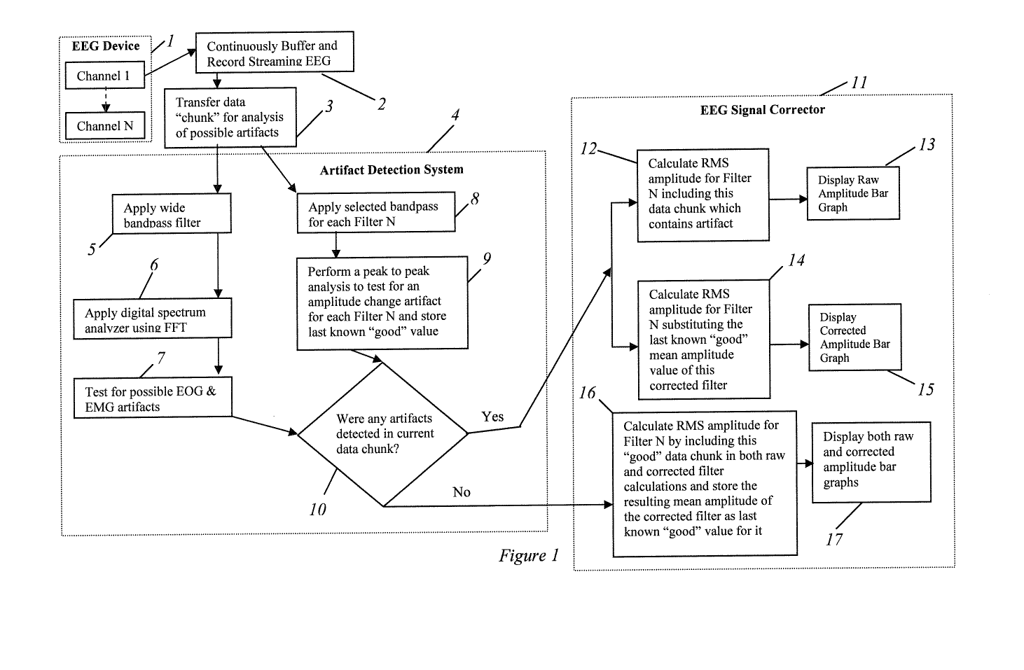

With reference to the flowchart of FIG. 1, the subject method comprises the identification of an artifact as delivered in a data stream from an EEG device. The device comprises a number of data streams, which is identified as element 1 of FIG. 1. In this disclosed method, the EEG feedback data signal stream from the one or more channels of the EEG device is sent by the EEG recording device to be continuously recorded and buffered 2. Small data samples or “chunks” of EEG data are simultaneously transferred 3 to an artifact detection system 4, which is comprised of two separate parts. The artifact detection system 4 analyzes the data chunks for possible artifacts. The first part of this artifact detection analysis consists of a wide bandpass filter 5. The filter, in one embodiment, uses the frequency range of 3 to 48 Hz, although other frequency ranges could be employed, if applicable to improve accuracy of the detection system or its speed of detection. Upon passing through the filter, a digital spectrum analyzer 6 using a fast Fourier transform (FFT) is applied. Testing of the FFT data occurs, as identified as element 7 in the flowchart. Based on different tests of the FFT data 7, it is possible to detect EOG artifacts, such as eye blinks and eye movements, and three types of EMG artifacts, including 1) raised eyebrows or forehead, 2) frowning, staring or squinting or 3) swallowing, grinning or temporomandibular joint tension.

The currently used settings for detecting EOG and EMG artifacts are based on threshold cutoff values for the amplitude, variance and ratios of the various bandwidths that are specified below for each type of artifact. These cutoff threshold values can be set to make the artifact detection system more or less sensitive in order to take into account the variability of the natural “baseline” muscular activity of each individual so that artifacts are not excessively reported or, conversely, not detected. The sensitivity settings were selected to optimize the accuracy of the artifact detection system by using cutoff thresholds over a designated range. The ranges of values can be empirically determined for each type of artifact.

In the detection of eye blinks and eye movements, the microvolt amplitude values for the cutoff thresholds that must be exceeded for the “low” bandwidth ranges from 25 to 50 microvolts. The microvolt amplitude values for the cutoff thresholds that must not be exceeded for the “high” bandwidth ranges from 15 to 45 microvolts. The variance over time threshold cutoff values to be exceeded for the eye blink signal varies from 5 to 40 milliseconds. In the case of the EMG artifact involving frowning and furrowing of the brow, the cutoff values of the microvolt amplitude for the “high” bandwidth must be greater than 9 to 27 microvolts. The EMG artifact involving staring or squinting is detected using ratios that vary from 1.3 to 2.3 based on the comparison of the variance in the percent values of the “high” to the “low” bandwidth ranges specified below. Swallowing, grinning, or temporomandibular joint tension uses cutoff threshold amplitude values that exceed microvolt levels ranging from 18 to 34 for the “high” bandwidth used. These value ranges and bandwidth ranges may be further defined by additional experimentation. The disclosed ranges include the best available data.

In further detail, eye blinks, eye movement, and the like are detected by detecting a spike (i.e., a sharp increase in amplitude) in a “low” bandwidth range of 4-13 Hz where eye blinks were empirically observed to occur when an elevation in amplitude is not simultaneously occurring in a “high” bandwidth of 36-48 Hz where EMG was empirically observed to occur. Eye blinks are detected based on cutoff EEG values for both amplitude values of the low and high ranges. For example, as explained above, the cutoff for the low range is in the range of 25 to 50 microvolts and for the high range it is less than a cutoff value in the range of 15 to 45 microvolts. The detection system also utilizes a cutoff score for the average variance over time of the eye blink signal using the 4-16 Hz bandwidth since it was empirically determined to be of shorter duration than EMG artifacts such as grinning. These cutoff values can be adjusted for each person's individual muscular characteristics.

In another example, frowning has been determined to manifest as a spike that is reflected in an overall increase in EEG activity throughout a “high” bandwidth of EEG of 33-48 Hz. By calculations, an EMG artifact associated with any frowning or furrowing or raised eyebrows is detected when forehead EMG is elevated by utilizing a cutoff score for the total variance of the signal activity of this type of artifact. This cutoff value can also be adjusted for each person's individual muscular characteristics.

In yet another example, the EMG artifact involving staring or squinting is detected using this subject method by the detection of a relatively higher spike (i.e., a sharp increase in amplitude) in a “high” bandwidth range of 26-48 Hz where generally only EMG activity is observed by comparing it to a “low” bandwidth range of 4-26 Hz where mostly EEG activity occurs. Again, calculations applied to the data stream are employed, as one of skill in the art will appreciate in light of this disclosure, to detect the EMG artifact associate when staring or squinting occurs. The calculations utilize a ratio cutoff score of the variance in percent values of the high bandwidth compared to variance in percent values of the low bandwidth for their signal amplitudes. This cutoff value can also be adjusted for each person's individual muscular characteristics.

In a still further example, swallowing, grinning or temporomandibular joint tension have been determined to manifest as a spike that is reflected in an overall increase in EEG activity predominately in the “high” bandwidth range of EEG from 33 to 48 Hz. Detection of the EMG artifact associated when swallowing, grinning, or temporomandibular joint tension occurs by utilizing a cutoff score for the amplitude of this type of artifact. Given that jaw muscles are much stronger than other facial muscles, the occurrence of a spike in the amplitude of the high bandwidth indicates this specific type of artifact. This cutoff value can also be adjusted for each person's individual muscular characteristics.

General artifact detection may also occur to detect any unusual change in the EEG amplitude caused by other types of EOG or EMG artifacts or an artifact resulting from environmental electrical noise (erg., an elevator motor starting). This type of artifact is detected by comparing the amplitude of the most recent EEG sample to the previous one. A cutoff score based on the difference of these two amplitude signal values is used to detect any sudden increase in the amplitude that would only result due to the occurrence of an artifact. This type of artifact is general in nature and does not need to be adjusted for a person's individual muscular differences. The system conducts a step of using pre-defined, universally applicable pattern recognition algorithms derived from the empirical observation of the natural and volitional occurrence of at least one relative amplitude ratio of at least one pattern of different EEG bandwidths for different types of possible EOG and EMG artifacts.

These above artifact detection methods can be enhanced by adding additional checks for other types of EMG activity that can generate artifacts that corrupt neurofeedback training. Also, as referential databases are developed, the sensitivity and accuracy of artifact detection by the subject method will increase. In addition, the adjustments for artifact sensitivity will be automated for each individual trainee.

The above artifacts may naturally occur simultaneously or in close temporal proximity during neurofeedback training. The method as disclosed herein analyzes each of these artifacts separately and, thus, one or more of these artifacts may be detected in any given EEG sample. This aspect of the artifact detection system makes it very sensitive, since as long as one artifact is detected, the EEG amplitude signal is corrected by a substitution method, as described further below. The methodology herein is also robust in the sense that various types of artifacts may overall or combine in their effect on the EEG signal amplitude and variance. The occurrence of any combination of artifacts does not impair the accuracy or sensitivity in the use of this method in identifying artifacts or in the correction of the EEG signal because whenever one or more artifacts are detected, the correction occurs by substitution of ‘good’ data and the correction does not require the measurement of the amplitude of the artifact signal to determine the “true” EEG signal (i.e., a ‘subtraction’ methodology).

In order to help accurately adjust the sensitivity of EOG and EMG artifacts, message prompts can be displayed that inform the trainer clinician when the presence of each type of artifact is detected (not illustrated). The trainer can then correctly adjust the sensitivity of each artifact detection filter. The goal is to set the sensitivity so as to not identify artifacts unless it is clear to the trainer that they are actually occurring based on an instruction set to the trainee to relax and the behavioral observations of the trainer during this time period. By this method, it would be possible to sample and mathematically set the artifact sensitivity and also to automatically adjust it during training. The completion of the EOG and EMG tests completes the first and more time-consuming part of the artifact detection system.

Additionally, message prompts are displayed to the trainee and the clinician alerting each to the detected presence of each type of artifact. The users can then respond by adjusting the sensitivity of each artifact detection filter or by reducing the amount of physical activity producing the artifact.

The artifact detection steps, identified as components 5-7 in FIG. 1, are performed simultaneously with components 8 to 9 using multi-threaded computer software coding techniques. Steps 5-7 and 8-9 could also be conducted independently. For the substitute methodology of the disclosed technique, it is not necessary to conduct both steps 5-7 and 8-9. Performing the combination of steps 5-7 and 8-9 simultaneously or independently acts as a catchall for all artifacts. However, one may choose to follow only one of the paths illustrated in FIG. 1 for the artifact detection system.

In this part of the artifact detection system, a selected bandpass filter is applied for each filter “N” individually 8. A peak-to-peak analysis is also applied individually to each selected bandpass filter from 1 to N, as illustrated by element 9 of the flowchart. This second independent part of the Artifact Detection System uses the peak-to-peak analysis 9 in order to identify any unusual increase or decrease in the signal. It is thought that an increase or decrease of two or more microvolts from the previous signal derived from the last known “good” value (i.e., the last artifact free signal detected) is unusual. This last test is used to detect any unusual changes in the EEG data sample that indicate it is likely to be the result of some unknown type of artifact. The actual amount of microvolt decrease or increase in step 9 that might be considered an artifact could be adjusted as needed. In this case the types of artifact would include any type that would result in a sudden change in EEG amplitude for a specific filter bandpass and, thus, is a final catchall provision for unspecified artifacts, such as spikes caused by environment factors (e.g., the trainee touching sensors or sensor wires) or EEG signal transmission errors.

Again, the sensitivity of this setting could later be improved by historical sampling of the data, adjustments based on statistical signal analysis and/or settings can be customized for specific filter bandpass ranges. Finally, a decision can be made as to whether any artifact was detected or not.

Either the first part of the artifact detection system (components 5-7 of Figure) or the second part (components 8-9 in FIG. 1) results in a decision as to whether any artifacts were detected or not at step 10 of FIG. 1. If any type of artifact was detected, then it is recorded and the system is set to mark a “True Artifact” for the specific data sample being analyzed.

The size of these data samples used in the Artifact Detection System 4 described above can range from a small number (e.g. four bytes) to a much larger number (e.g., 1,024 bytes or more). The size of these chunks is determined by the maximum data sample sent by the EEG recording device. The number of the data sample can be fixed in order to minimize the detection of artifacts and/or to increase the sensitivity of artifact detection or to facilitate the specific detection of certain types of artifacts. Also, based on an individual's EEG pattern, different artifacts can be targeted for more accurate and specific analysis. The number of bytes in the data sample can be changed to make the artifact detection system faster by using more complex statistical analysis or turning off artifact detection checks that are not found to occur frequently enough to warrant ongoing detection during that session or for that specific individual. Also, it is thought that wide bandpass filter 5 in FIG. 1 is optional and/or could incorporate the separate peak-to-peak analysis as part of the digital spectrum analyzer 6 in order to make the artifact detection system faster. Techniques that lead simplify or speed up the artifact detection system are thought to fall within the scope of the claims, as provided below.

Five types of artifacts that can contaminate EEG data have been identified. Identification will be more accurate where additional statistical signal analysis occurs and/or when more types of artifacts that can corrupt EEG data have been identified. The subject method is customizable and adjustable in order to account for the types of artifacts identified for specific individuals based on a statistical analysis of their EEG patterns under a variety of different assessment conditions (e.g., reading, writing, taking a test, etc.). Also, the types of artifacts may be found to manifest differently or require different sensitivity settings for different areas of the brain or for different individuals. The subject method will become more effective as additional statistical analysis and identification features are incorporated into the method. The method will further increase in accuracy, sensitivity and effectiveness as these components are incorporated.

This artifact detection and correction system could also be implemented in EEG recording devices before any signal is sent for computer analysis. In this embodiment, the firmware of the EEG recording device would incorporate the subject method.

Following artifact detection and the identification of the presence of an artifact, data is passed to an EEG Signal Corrector 11. In the case that the artifact detection system does not detect any artifacts, then the data sample is used in the computations of the RMS amplitude 16 by including this “good” data chunk in both raw and corrected filter calculations. The resulting mean amplitude of the corrected or uncorrupted filter is stored as the last known “good” value for this filter. The raw and corrected amplitude bar graphs are displayed 17. The bar graphs are continuously updated in real-ime.

Bar graphs and audio sounds are used to provide feedback of the raw and corrected EEG signals, but other types of feedback including tactile, reward or spoken words could be used. The calculations for the feedback can also vary and may include feedback for variance, ratio, correlation, coherence or complex multi-modal feedback based on the amplitude or other statistical aspects of the corrected EEG signal data analysis. The data analysis uses a 256 byte, one second data sample in computing the amplitude strength of the specified EEG filter band. However, the data sample can be customized as desired.

The data sample generated by the EEG device 1 is typically smaller in size and is usually in the range of 16 to 64 bytes of data. This more recent data sample is copied to the data array used to compute the feedback bar graph amplitude and the least recent data is discarded.

In the case where an artifact is detected in the most recent data sample at element 10 of FIG. 1, the data sample is also provided to the EEG signal corrector 11 in order to calculate the RMS amplitude. In this case, however, two RMS amplitudes are calculated. First, signal corrector 11 calculates the RMS amplitude for filter N for the data chunk containing the detected artifact 12. Simultaneously, corrector 11 calculates the RMS amplitude for filter N substituting the last known “good mean amplitude value for this corrected filter 14. The RMS amplitude for the uncorrected data chunk is displayed in the raw amplitude bar graph 13. The corrected RMS amplitude is displayed in the corrected amplitude bar graph 15. Only one version of graphs 13, 15, and 17 are displayed at any one time and always display the most recent data sample/chunk calculated at steps 12, 14 and 16.

In further detail for correction step 14, the correction is computed by substituting into the 256-byte array of the corrected filter the last known “good” mean amplitude value for the corrected filter. In this way the best estimate of the current EEG RMS amplitude is calculated. As part of this process for the corrected filter 14, each byte in the data sample identified as containing contaminated data is set to this last known good value and this corrected data sample is used in computing the corrected filter; replacing the same number of bytes that are the least recent. The actual artifact data sample is still used in computing the raw RMS amplitude 12, as the raw filter is supposed to reflect the presence of an artifact. Finally, after correction the feedback in the current form of a bar graph is displayed for both raw and corrected filters.

The entire process of the artifact system continuously repeats. The time required for the artifact detection and correction can take between 256 to 512 milliseconds and is generally less than 350 milliseconds. The variance is due to the type of filters used and the number of artifacts detected. Additional filter or processing may impact the processing time while faster processers may offset any additions. FIR filters are used for the bandpass, but IIR or other types could be substituted in order to speed up the bandpass filters. It is also possible to speed up or use a different digital spectrum analysis or have these calculations pre-calculated in the EEG hardware device. Also, to smooth out and calculate the EEG signal a Hilbert transform is used as part of an envelope detector in order to obtain the most accurate calculation of signal amplitude for each filter. Other types of transforms and envelope detectors are possible to use and may make this system faster or more accurate.

During normal operation (i.e. when no artifacts are detected), the corrected amplitude bar graph's output is identical to the raw amplitude bar graph 16, 17. However, during an EEG artifact event, the corrected amplitude graph's movement 14, 15 is corrected to prevent the user from seeing a misleading value. Rather than instantly freezing the envelope detector's output, the envelope detector is gradually damped causing the bar graph to settle at the last known “good” amplitude value. This means that the corrected amplitude bar graph has its own envelope detector system separate from the raw amplitude bar graph.

It is thought that the subject artifact detection and correction system and method further applies to:
1. Use in developing a normative quantitative EEC database that would be more accurate and would allow data to be collected while those being assessed were engaging in active learning exercises such as cognitive training tasks, psychological tests, occupational tasks or academic work.
2. The application of artifact detection and correction to coherence training.
3. Detection and correction of artifacts inherent in reading tasks, so that neurofeedback could be used to more quickly improve brainwave activity associated with better or improved reading skills.
4. More adjustable levels of sensitivity for each of the artifact filters or more types of artifacts and customization of the automatic adjustment of sensitivity levels automatically for each individual.
5. Future reduction of the computational time needed to perform the artifact detection analyses and filtering.
6. Recordation of the characteristics of each detected artifact for possible artificial intelligence learning and the development of an artifact database using age, sex, clinical history and brain location site as factors.
7. Peak performance training to help athletes improve their performance in various sports, such as golf, can also be enhanced by optimizing this artifact detection and correction system in order to specifically control for any large muscle movement artifact that may occur during neurofeedback training.

The above disclosure describes a method for simultaneously and concurrently identifying and quantifying a wide variety of types of artifacts including facial electromyographic (EMG) and eye movement electrooculargraphic (EOG) activity, which naturally contaminate electroencephalographic (EEG) waveforms and, consequently impair the neurofeedback learning process. This disclosure provides a number of new, unique and non-obvious methods not used in prior art to make neurofeedback training easier and more effective by identifying and eliminating in real-time the inclusion of artifacts that naturally and spontaneously corrupt the accurate measurement of EEG signal data needed for effective neurofeedback training, particularly in frontal and temporal lobes of the brain. This multi-level, universally applicable, pre-defined pattern recognition artifact detection and correction system provides a method for enhancing EEG biofeedback training by detecting and eliminating any brief contaminated epoch of EEG activity from being included in the calculation and analysis of the EEG signal; making it possible to provide without any interruption visual, auditory and/or tactile feedback of a “true” EEG signal that through operant conditioning learning principles enables individuals to more quickly and easily learn to control their brainwave activity using neurofeedback. It is apparent from the nature of this invention that while specific forms have been illustrated and described, various improvements and modifications can be made within its spirit and scope. Thus, it is not intended that this invention in this sense be limited in any way, except as specified in the appended claims.

What is claimed is:

1. A method for identifying and correcting the occurrence of any of a wide variety of different types of concurrent EOG and/or EMG artifacts of one or more EEG signals during EEG monitoring of an individual, the method comprising
collecting each of one or more individual signals from one or more specified EEG frequency bandwidths;
passing the signal as a data sample to an artifact detection system, the artifact detection system
applying a first wide bandpass filter to the data sample;
applying a digital spectrum analyzer to the filtered data sample;
testing for possible EOG and EMG artifacts; and
applying selected bandpass for each filter;
performing analysis to test for an artifact for each filter;
storing the last known value that does not represent an artifact;
determining whether an artifact is present in the data sample;
in the event an artifact is detected, substituting the last known value that does not reflect an artifact for the data containing the artifact;
forwarding the data sample to a signal corrector, the signal corrector calculating RMS amplitude; and
displaying a raw amplitude graph and a corrected amplitude graph.

2. The method of claim 1, further comprising the step of setting cutoff thresholds used in determining the occurrence of EOG artifacts to take into account the different signal strengths of the amplitude and variance of EOG activity in different bandwidths involving the naturally spontaneous and volitionally occurring activity of the monitored individual.

3. The method of claim 1, further comprising the step of setting cutoff thresholds used in determining the occurrence of EOG artifacts to take into account the different signal strengths of the amplitude and variance of EOG activity in different bandwidths of the monitored individual, the activity selected from the group comprising eye blinks, eye strain, eye closure and eye movement.

4. The method of claim 1, further comprising the step of setting cutoff thresholds used in determining the occurrence of EMG artifacts to take into account the different signal strengths of the amplitude, a ratio and variance of EMG activity in different bandwidths related to the naturally spontaneous and volitionally occurring activity of the monitored individual.

5. The method of claim 1, further comprising the step of setting cutoff thresholds used in determining the occurrence of EMG artifacts to take into account the variance of different signal strengths of the amplitude of EMG activity in a specific bandwidth of the monitored individual, the activity selected from the group comprising raising of the individual's eyebrows and furrowing of the individual's brow.

6. The method of claim 1, further comprising the step of setting cutoff thresholds used in determining the occurrence of EMG artifacts to take into account the ratio of the variance of the different signal strengths of the amplitude of EMG activity in different bandwidths related to the naturally spontaneous and volitionally occurring activity of the monitored individual, the activity selected from the group comprising squinting by the individual and straining of the muscles which surround the individual's eyes.

7. The method of claim 1, further comprising the step of setting cutoff thresholds used in determining the occurrence of EMS artifacts to take into account the different signal strengths of the amplitude of EMG activity for a specific bandwidth related to the naturally spontaneous and volitionally occurring activity of the temporomandibular joint muscles.

8. The method of claim 1, further comprising the steps of identifying and correcting the EEG signal in real-time for any sudden or significant change in the amplitude or coherence of any one of more EEG signals during a brief epoch that does not naturally occur in the EEG waveform based on a cutoff threshold in order to take into account the occurrence of distortions and artifacts that could possibly occur related to external environmental electrical signals, EOG, EMG or other types of physiologically or environmentally based electrical signal activity.

9. The method of claim 1, wherein the step of applying a digital spectrum analyzer to the filtered data sample further comprises the step of analyzing the EEG signals using a Fast Fourier transform in order to compute signal amplitude values, variances and a ratio of the amplitude values or signal variances for specified bandwidths that are used in determining whether predetermined threshold values are exceeded that indicate the occurrence of an artifact.

10. The method of claim 1, further comprising the step of using pre-defined, universally applicable pattern recognition algorithms derived from the empirical observation of the natural and volitional occurrence of the at least one relative amplitude, ratio or variance of at least one pattern of different EEG bandwidths for different types of possible EOG and EMG artifacts.

11. A method for identifying and correcting the occurrence of any of a wide variety of different types of concurrent EOG and/or EMS artifacts of one or more EEG signals during EEG monitoring of an individual, the method comprising
collecting each of one or more individual signals from one or more specified EEG frequency bandwidths;
passing the signal as a data sample to an artifact detection system, the artifact detection system applying a first wide bandpass filter to the data sample;
applying a digital spectrum analyzer to the filtered data sample;
testing for possible EOG and EMG artifacts;
determining whether an artifact is present in the data sample;
in the event an artifact is detected, substituting the last known value that does not reflect an artifact for the data containing the artifact;
forwarding the data sample to a signal corrector, the signal corrector calculating RMS amplitude; and
displaying a raw amplitude graph and a corrected amplitude graph.

12. The method of claim 11, further comprising the step of setting cutoff thresholds used in determining the occurrence of EOG artifacts to take into account the different signal strengths of the amplitude and variance of EOG activity in different bandwidths involving the naturally spontaneous and volitionally occurring activity of the monitored individual.

13. The method of claim 11, further comprising the step of setting cutoff thresholds used in determining the occurrence of LOG artifacts to take into account the different signal strengths of the amplitude and variance of EOG activity in different bandwidths of the monitored individual, the activity selected from the group comprising eye blinks, eye strain, eye closure and eye movement.

14. The method of claim 11, further comprising the step of setting cutoff thresholds used in determining the occurrence of EMG artifacts to take into account the different signal strengths of the amplitude, a ratio and variance of EMG activity in different bandwidths related to the naturally spontaneous and volitionally occurring activity of the monitored individual.

15. The method of claim 11, further comprising the step of setting cutoff thresholds used in determining the occurrence of EMG artifacts to take into account the variance of different signal strengths of the amplitude of EMG activity in a specific bandwidth of the monitored individual, the activity selected from the group comprising raising of the individual's eyebrows and furrowing of the individual's brow.

16. The method of claim 11, further comprising the step of setting cutoff thresholds used in determining the occurrence of EMG artifacts to take into account the ratio of the variance of the different signal strengths of the amplitude of EMG activity in different bandwidths related to the naturally spontaneous and volitionally occurring activity of the monitored individual, the activity selected from the group comprising squinting by the individual and straining of the muscles which surround the individual's eyes.

17. The method of claim 11, further comprising the step of setting cutoff thresholds used in determining the occurrence of EMG artifacts to take into account the different signal strengths of the amplitude of EMG activity for a specific bandwidth related to the naturally spontaneous and volitionally occurring activity of the temporomandibular joint muscles.

18. The method of claim 11, wherein the step of applying a digital spectrum analyzer to the filtered data sample further comprises the step of analyzing the EEG signals using a Fast Fourier transform in order to compute signal amplitude values, variances and a ratio of the amplitude values or signal variances for specified bandwidths that are used in determining whether predetermined threshold values are exceeded that indicate the occurrence of an artifact.

19. The method of claim 11, further comprising the step of using pre-defined, universally applicable pattern recognition algorithms derived from the empirical observation of the natural and volitional occurrence of the at least one relative amplitude, ratio or variance of at least one pattern of different EEG bandwidths for different types of possible EOG and EMG artifacts.

20. A method for identifying and correcting the occurrence of any of a wide variety of different types of concurrent EOG, EMI and/or environmentally generated artifacts of one or more EEG signals during EEG monitoring of an individual, the method comprising
collecting each of one or more individual signals from one or more specified EEG frequency bandwidths;
passing the signal as a data sample to an artifact detection system, the artifact detection system applying a selected bandpass for each filter;
performing analysis to test for an artifact for each filter;
storing the last known value that does not represent an artifact;
determining whether an artifact is present in the data sample;
in the event an artifact is detected, substituting the last known value that does not reflect an artifact for the data containing the artifact;
forwarding the data sample to a signal corrector, the signal corrector calculating RMS amplitude; and
displaying a raw amplitude graph and a corrected amplitude graph.

21. The method of claim 20, further comprising the steps of identifying and correcting the EEG signal in real-time for any sudden or significant change in the amplitude or coherence of any one of more EEG signals during a brief epoch that does not naturally occur in the EEG waveform based on a cutoff threshold in order to take into account the occurrence of distortions and artifacts that could possibly occur related to external environmental electrical signals, EOG, EMG or other types of physiologically or environmentally based electrical signal activity.
